(19) United States
(12) Patent Application Publication
(10) Pub. No.: US 2026/0295202 A1
(43) Pub. Date: Oct. 1, 2026

(54) SOOTHING DEVICE

(71) Applicant: NINGBO SINCERE LEISURE PRODUCTS CO., LTD., Ningbo (CN)

(72) Inventor: Wenguang LU, Ningbo (CN)

(21) Appl. No.: 19/260,625

(22) Filed: Jul. 7, 2025

(30) Foreign Application Priority Data
Apr. 1, 2025 (CN) 202510405314.8

(51) Int. Cl. A61M 21/02; A61M 21/00

(52) U.S. Cl. CPC A61M 21/02; A61M 2021/0022; A61M 2021/0061; A61M 2205/3584; A61M 2205/8206; A61M 2205/8237; A61M 2230/06; A61M 2250/00

(57) ABSTRACT

A soothing device includes: a stepless adjustment structure, configured to receive the operation instruction of the user and realize different frequency adjustments of the soothing device according to the operation intention of the user; and a soothing structure, comprising a control board and a vibration module, where the control board is respectively connected to the stepless adjustment structure and the vibration module, and is configured to control the vibration module to vibrate, stop vibration, and change of vibration frequency.

### CROSS-REFERENCE TO RELATED APPLICATIONS

This application claims priority to Chinese Patent Application No. 202510405314.8, filed on Apr. 1, 2025, the content of which is incorporated herein by reference in its entirety.

### TECHNICAL FIELD

The present application relates to the technical field of pet products, more particularly to a soothing device.

### BACKGROUND

With the improvement of people's living standards, the pet market is increasingly prosperous, and many families like to keep pets. People tend to build deep bonds during the time they spend with their pets and regard their pets as part of their family. Different people or animals have different needs. For example, different heartbeat states will exist in different environments. Pets often lose interest in toys because of a lack of function. Therefore, there is an urgent need for a soothing device to solve the technical problem.

### SUMMARY

An object of the present disclosure is to provide a soothing device which can effectively soothe the uneasy emotions of humans and animals and meet the needs of different individuals.

According to a first aspect, the embodiment of the present disclosure provides a soothing device comprising: a stepless adjustment structure configured to receive an operation instruction of a user and realize different frequency adjustment of the soothing device according to an operation intention of the user; and a soothing structure comprising a control board and a vibration module, where the control board is respectively connected to the stepless adjustment structure and the vibration module, and is configured to control the vibration module to vibrate, stop vibration, and change of vibration frequency.

In the above implementation, the stepless adjustment structure and the vibration module are connected to the control board. When the soothing device is used for a pet, the stepless adjustment structure receives the instruction of the user and converts the instruction into a stepless adjustment signal. The control board processes the stepless adjustment signal and outputs the signal to the vibration module, so that the vibration module starts to vibrate, adjust the vibration frequency or stops vibrating, etc., so as to simulate the corresponding frequency and realize the stepless adjustment, which can effectively soothe the uneasy emotions of animals and meet the needs of different individuals.

In some embodiments, the stepless adjustment structure comprises a switch and a stepless converter. The switch is connected to the stepless converter. The switch is provided with an operation portion. The stepless converter is configured to receive an action applied by the user to the operation portion and convert the action into the corresponding stepless adjustment signal.

In the above implementation, the switch is connected to the stepless converter. The user feeds back the needs through the switch to realize the control of turning on or off. Also, under the action of the stepless converter, the heartbeat frequency can be adjusted steplessly to meet the needs of different individuals and effectively soothe uneasy emotions.

In some embodiments, the switch comprises an adjustment knob to adjust along a first direction or a second direction. The adjustment knob is configured to turn on and adjust the vibration frequency of the vibration module when rotated along the first direction. The adjustment knob is configured to turn off and/or adjust the vibration frequency of the vibration module when rotated along the second direction. The first direction is opposite to the second direction.

In the above implementation, the adjustment knob can be rotated along the first direction and the second direction to realize the control of turning on or off. Also, the heartbeat frequency can be adjusted steplessly, which simplifies the structure and meets the needs of different individuals.

In some embodiments, the stepless adjustment structure comprises a remote device and a wireless connection module. The remote device communicates with the wireless connection module. After the wireless connection module receives a communication command from a user, the control board instructs the vibration module to operate based on the communication command.

In the above implementation, the remote device communicates with the wireless connection module. The user outputs an instruction through the remote device, and after the wireless connection module receives the communication instruction, the control board controls the vibration module to perform the corresponding operation. It is beneficial for users to effectively soothe their pets when users cannot accompany their pets all the time, which improves the applicability of the products.

In some embodiments, the stepless adjustment structure comprises a sensor. The sensor is connected to the control board to collect information and provide corresponding frequency signals to the control board.

In the above implementation, the sensor is configured to sense the information of the pet and feed back to the control board, so as to provide the corresponding vibration frequency under the action of the vibration module, realize stepless adjustment, and meet the needs of different individuals.

In some embodiments, the soothing device comprises a mounting shell. The mounting shell has a receiving cavity. The receiving cavity is configured to receive the soothing structure and the stepless adjustment structure. The receiving cavity can receive the soothing structure and the stepless adjustment structure, reduce external damage to the structure, and improve the service life of the structure.

In some embodiments, the soothing device further comprises a timing structure. The timing structure is connected to the control board, and at least a portion of the timing structure is exposed to the mounting shell.

In the above implementation, after the soothing device is turned on, the stepless adjustment structure is adjusted steplessly, so that the control board controls the vibration module to perform corresponding actions. The timing structure can control the soothing device to work within a fixed duration to realize the timing function and meet different functional requirements.

In some embodiments, the soothing device further comprises a battery structure. The battery structure connects the stepless adjustment structure and the soothing structure, and is configured to provide electrical energy.

In the above implementation, by connecting the battery structure to the stepless adjustment structure and the soothing structure respectively, the battery structure can be provided in a built-in manner to avoid frequent replacement and be more environmentally friendly.

In some embodiments, the soothing device further comprises a charging interface. The charging interface is connected to the control board, to charge the battery structure, to avoid frequent replacement to be more environmentally friendly.

In some embodiments, the soothing device further comprises an indicator light. The indicator light is connected to the control board, which can indicate, conveniently remind the user of relevant information and improve the functionality of the product.

Other features and advantages of the present disclosure will be set forth in the following specification, or some of the features and advantages can be inferred or unquestionably determined from the specification, or can be learned by practicing the above-described techniques of the present disclosure.

In order to make the above objects, features, and advantages of the present disclosure more readily understood, preferred embodiments will be described in detail below with reference to the accompanying drawings.

### BRIEF DESCRIPTION OF THE DRAWINGS

In order to more clearly explain the technical solutions of the embodiments of the present disclosure, the following drawings that need to be used in the embodiments of the present disclosure will be briefly introduced. It should be understood that the following drawings only illustrate certain embodiments of the present disclosure, and therefore should not be regarded as limiting the scope. For those skilled in the art, other related drawings can be obtained from these drawings without making inventive labor.

FIG. 1 is a schematic structural diagram of a soothing device according to an embodiment of the present disclosure.

FIG. 2 is an exploded view of the soothing device according to the embodiment of the present disclosure.

### DETAILED DESCRIPTION OF THE EMBODIMENTS

Hereinafter, the technical solutions in the embodiments of the present disclosure will be clearly and completely described with reference to the accompanying drawings in the embodiments of the present disclosure, and it is obvious that the described embodiments are only a part of the embodiments of the present disclosure, but not all the embodiments. The components of the embodiments of the present disclosure generally described and illustrated in the drawings herein can be arranged and designed in a variety of different configurations. Accordingly, the following detailed description of embodiments of the application provided in the accompanying drawings is not intended to limit the scope of the claimed application, but is merely to represent selected embodiments of the application. Based on the embodiments of the present disclosure, all other embodiments obtained by those skilled in the art without making creative work belong to the scope of protection of the present disclosure.

At present, as an important member of personal or family life and a supplement to affection, more and more people like to keep pets. However, usually due to busy work, users can't accompany their pet 24 hours or for a long time, which leads to the adverse consequences of pets feeling lonely and depressed. Therefore, the pet toys are created. At present, there are many kinds of pet toys on the market, but most toys only pay attention to the training function or beautiful appearance design, and lack soothing for pets.

In the implementation, the inventor found that different animals have different comfort zones for heartbeat states. The same animal will have different comfort zones in different situations. For example, one heartbeat state is required when sleeping at night, and another heartbeat state is needed when getting sick. The heartbeat state comprises the duration of a single heartbeat and the frequency of a single heartbeat. Due to the single function of the conventional simulation heartbeat products in the market, it is easy for pets to lose interest quickly during use. The conventional product technology has neither an adjustable solution for heartbeat frequency nor a specific working timing adjustment function.

For another similar application scenario, the study found that the soothing heartbeat sound of infants and babies is a method to help infants and babies feel safe and relaxed by using heartbeat sounds similar to those heard in the mother's body during fetal development. However, different human individuals have more obvious differences in soothing effects on different frequencies of heartbeats. The conventional product technologies also do not have an adjustable solution for the heartbeat frequency.

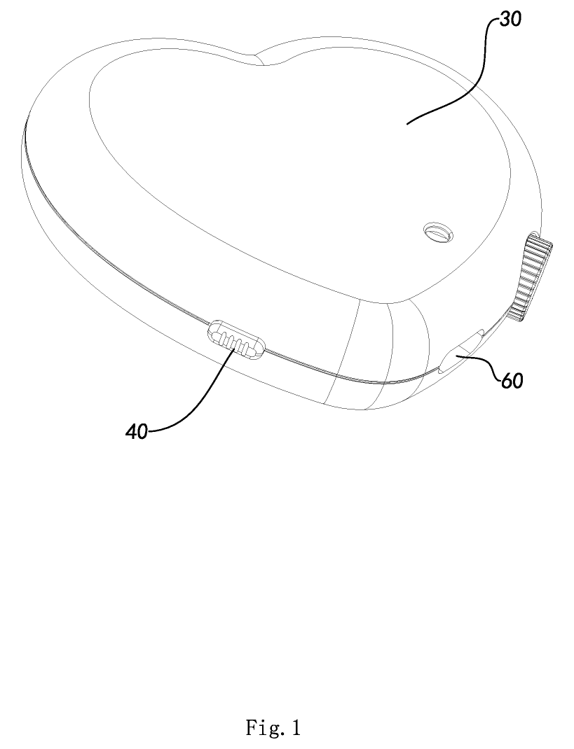

In view of the above, as shown in FIGS. 1 - 2, in the first aspect, an embodiment of the present disclosure provides a soothing device comprising a stepless adjustment structure 10 and a soothing structure 20. The stepless adjustment structure 10 is connected to the soothing structure 20, and the stepless adjustment structure 10 can perform stepless adjustment after receiving an operation instruction from a user, so that the soothing structure 20 can vibrate at a corresponding frequency and finally realize soothing to pets or infants.

Specifically, the stepless adjustment structure 10 is configured to receive the operation instruction of the user and realize different frequency adjustments of the soothing device according to the operation intention of the user. The soothing structure 20 comprises a control board 201 and a vibration module 202. The control board 201 is respectively connected to the stepless adjustment structure 10 and the vibration module 202 and is configured to control the vibration module 202 to vibrate, stop vibration, and change in vibration frequency according to the stepless adjustment signal.

Exemplarily, the operation instruction of the user can be a mechanical operation instruction. That is, the user performs mechanical adjustment by manually adjusting (mainly taking a rotational action) the stepless adjustment structure 10. The stepless adjustment structure 10 is connected to the control board 201. After the stepless adjustment structure 10 is manually rotated and adjusted by the user, the control board 201 detects that the soothing device is turned on or/and the stepless adjustment structure 10 is shifted to a different resistance value position based on the operation instruction of the user, so the control board 201 instructs the vibration module 202 to start to vibrate or/and vibrate at the operating frequency corresponding to the current resistance value position. In this way, the heartbeat frequency can be changed according to the needs of the user, and this change is arbitrary (so-called stepless adjustment) and is not limited to a few cases in the related arts. Based on different resistance value positions of the stepless adjustment structure 10, the soothing device correspondingly generates different heartbeat frequencies, thereby completing stepless adjustment of the heartbeat frequency. Instead of actively detecting and controlling by the control board 201, the overall drive circuit can automatically adjust the vibration frequency directly based on the adjustment result of the stepless adjustment structure 10, thereby realizing a simplified adjustment circuit.

It can be understood that the control board 201 comprises at least a circuit board, and the vibration module 202 comprises, but is not limited to, a vibration motor.

In the above implementation, the stepless adjustment structure 10 and the vibration module 202 are connected to the control board 201. When the soothing device is used for a pet, the stepless adjustment structure 10 receives the instruction of the user and converts the instruction into a stepless adjustment signal. The control board 201 processes the stepless adjustment signal and outputs the signal to the vibration module 202, so that the vibration module 202 starts to vibrate, adjust the vibration frequency or stops vibrating, etc., so as to simulate the corresponding frequency and realize the stepless adjustment, which can effectively soothe the uneasy emotions of animals and meet the needs of different individuals.

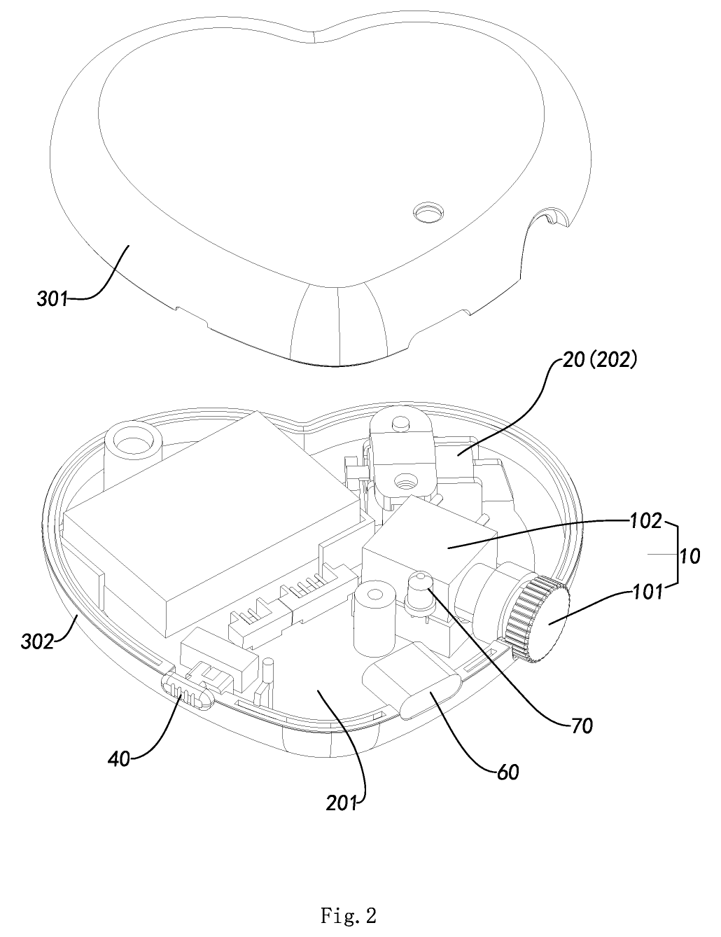

As shown in FIG. 2, the stepless adjustment structure 10 comprises a switch 101 and a stepless converter 102. The switch 101 is connected to the stepless converter 102. The switch 101 is provided with an operation portion. The stepless converter 102 is configured to receive an action applied by the user to the operation portion and convert the action into the corresponding stepless adjustment signal.

Exemplarily, the switch 101 comprises, but is not limited to, an adjustment knob. The stepless converter 102 can be a potentiometer, a rotary encoded switch, or the like. When need to operate, the user can manually rotate the adjustment knob to rotate the potentiometer to different resistance positions. If a certain rotation angle threshold is exceeded, the control board 201 can detect and interpret it as a turn-on signal. The control board 201 can instruct the vibration module 202 to start operation at the operating frequency corresponding to the resistance position to generate a heartbeat effect. If the operating frequency needs to be adjusted, the user only needs to continue to rotate the adjustment knob, so that different vibration frequencies are generated based on resistance values at different positions, to realize the stepless adjustment.

In some embodiments, the switch 101 comprises the adjustment knob to adjust along a first direction or a second direction. When the adjustment knob is rotated in the first direction, the adjustment knob is configured to turn on and/or adjust the vibration frequency of the vibration module 202. When the adjustment knob is rotated in the second direction, the adjustment knob is configured to turn off and/or adjust the vibration frequency of the vibration module 202. The first direction is opposite to the second direction.

Exemplarily, the first direction comprises, but is not limited to, a clockwise rotational direction of the adjustment knob, and the second direction comprises, but is not limited to, a counterclockwise rotational direction. Therefore, when needs to operate, firstly the switch 101 is rotated clockwise, and after the control board 201 detects the turn-on signal, the control board 201 instructs the vibration module 202 to work at a certain operating frequency to generate the heartbeat effect. If the switch 101 is continuously rotated clockwise, the control board 201 detects a different resistance value generated based on the adjustment of the switch 101. Therefore, the control board 201 instructs to control the vibration module 202 to operate at the corresponding operating frequency. In this way, the heartbeat frequency changes accordingly. The different resistance values correspondingly generate different heartbeat frequencies, and the change of resistance values is basically continuous, so that the adjustment of the heartbeat frequency can be a stepless adjustment. It can be understood that the soothing device is turned on when rotating clockwise, and the greater the clockwise angle, the higher the corresponding heartbeat frequency, and vice versa. If rotating counterclockwise to the initial position, it can be configured to turn off the soothing device.

In some embodiments, the inventor considers the rapid development of wireless technology and the widespread popularity of smart homes, and the user's adjustment can be either mechanical instructions (for example, human hands directly operate the soothing device) or communication instructions (for example, voice recognition, image recognition, or remote control, etc.).

For example, if the user adjustment needs to realize control and adjustment of the soothing device through wireless communication instructions, the stepless adjustment structure 10 can further comprise a remote device and a wireless connection module. The user can communicate with the wireless connection module through the remote device, and after the wireless connection module receives a specific communication instruction from the user, the control board 201 instructs the vibration module to turn on, turn off, or adjust vibration based on the communication instruction of the user, thereby realizing stepless adjustment.

Exemplarily, the remote device comprises a terminal hardware, being a physical device, comprising a computer, a smart home, a tablet, a remote controller, or a smartphone. These devices are connected to the soothing structure 20 via a wired or wireless connection module through a built-in communication interface and corresponding software, enabling remote access and control.

In the above implementation, if the use of non-mechanical user instructions (such as communication instructions) is involved, then the stepless converter 102 is sometimes not necessary, as long as the instruction signal of stepless adjustment can be sent by the remote device.

However, sometimes, in order to take into account the needs of different users, and considering the general stability of remote communication command adjustment, two control manner of mechanical command and communication command can be provided in the soothing device. In this way, not only can general mechanical contact adjustment be realized, but also wireless non-contact adjustment can be realized. In this case, if the user is at home or close to the pet, manual mechanical adjustment can be selected so that the control board 201 controls the vibration module 202 to perform corresponding actions. If the user goes out or is far away from the pet, he or she can select the remote communication control and adjustment. In this way, different choices are beneficial for users to effectively soothe pets when they cannot accompany them 24 hours or for a long time, and improve the applicability of the products.

Exemplarily, it will be appreciated that the remote device is sometimes not necessary to enable the reception of a remote communication instruction. For example, the soothing device can be provided with a voice or image recognition module configured to receive a voice command or a remote action command from the user, and to control the vibration module 202 to adjust the vibration frequency according to the user's intention, without requiring the user to approach the soothing device for manual adjustment or remote adjustment by a specific remote adjustment device.

In some embodiments, the stepless adjustment structure 10 can further comprise a sensor connected to the control board 201 to collect information and provide corresponding frequency signals to the control board 201. The stepless converter 102 is connected to the control board 201. The sensor comprises, but is not limited to, a sensing device, for example, sensing a heartbeat sound of a pet, and providing a corresponding vibration frequency according to the received pet information.

Further, the soothing device can be provided with a storage device to pre-store a suitable pet vibration frequency data for quick call by the user. It can also be used in combination with the sensing device to collect and analyze the pet's heartbeat data and filter the heartbeat signal to obtain a low-frequency signal. Finally, the imitation heartbeat vibration signal in the low-frequency signal is stored. In the process of soothing the pet with the soothing device, the control board 201 can output an imitation heartbeat vibration signal to the vibration module 202 so that the vibration module 202 vibrates at the heartbeat frequency.

As shown in FIGS. 1 and 2, the soothing device comprises a mounting shell 30 configured with a receiving cavity configured to receive the soothing structure 20 and the stepless adjustment structure 10. The receiving cavity can receive the soothing structure 20 and the stepless adjustment structure 10, reduce external damage to the structure, and improve the service life of the structure.

For example, the mounting shell 30 comprises an upper shell 301 and a lower shell 302, and the upper shell 301 and the lower shell 302 can be fixed in a buckle manner or can be fixed by a bolt. The shape of the mounting shell 30 can be a heart shape, a circle shape, or the like, and is not specifically limited here.

In some embodiments, the soothing device further comprises a timing structure 40 comprising, but not limited to, a timing switch. The timing structure 40 is connected to the control board 201, and at least a part of the timing structure 40 is exposed to the mounting shell 30 to facilitate the user to perform the timing operation thereof.

In the above implementation, after the soothing device is turned on, the stepless adjustment structure 10 performs stepless adjustment, so that the control board 201 controls the vibration module 202 to perform corresponding actions. The timing structure 40 can control the soothing device to operate within a fixed duration to realize the timing function and meet different functional requirements.

As shown in FIG. 2, the soothing device further comprises a battery structure 50 connecting the stepless adjustment structure 10 and the soothing structure 20 and configured to provide electrical energy. For example, the battery structure 50 is disposed in the receiving cavity of the mounting shell 30. In order to achieve fixation of the battery structure 50, the receiving cavity can be provided with a fixing frame. The fixing frame is L-shaped and is arranged at the diagonal positions of the battery structure 50. Of course, in other embodiments, other fixing manners can also be used to fix the battery structure 50.

The battery structure 50 comprises, but is not limited to, a lithium battery. The battery structure 50 can be a rechargeable battery or a disposable battery.

In the above implementation, by connecting the battery structure 50 to the stepless adjustment structure 10 and the soothing structure 20 respectively, the battery structure 50 can adopt a built-in manner to avoid frequent replacement and be more environmentally friendly.

In some embodiments, the soothing device further comprises a charging interface 60 comprising, but not limited to, type C. The charging interface 60 is connected to the control board 201, and a part of the charging interface 60 is exposed to the mounting shell 30 to charge the battery structure 50, to avoid frequent replacement to be more environmentally friendly.

As shown in FIGS. 1 and 2, the soothing device further comprises an indicator light 70, the indicator light 70 is connected to the control board 201, and the indicator light 70 is exposed on the upper shell 301. It is understood that the indicator light 70 can indicate a lack of power or sufficient power of the battery structure 50, or the like, and can also indicate a related vibration pattern. The indicator light 70 indicates the function, which is convenient to remind users of relevant information and improves the functionality of the product.

As for the description of the application scenario of the above soothing device, the soothing device can be used together with toys for pets or infants. The toy can be a puppet, a pillow, a bed, or the like, and the connection manner thereof can be a fixed type or a detachable type. For example, buckle type and bundle type. Here will not enumerate.

The above is merely a specific embodiment of the present disclosure, but the scope of protection of the present disclosure is not limited thereto, and any person skilled in the art can easily think of changes or substitutions within the technical scope disclosed in the present disclosure, and should be covered within the scope of protection of the present disclosure. Therefore, the scope of protection of the present disclosure should be the same as that of the claims.

What is claimed is:

1. A soothing device, comprising:
a stepless adjustment structure, configured to receive the operation instruction of a user and realize different frequency adjustments of the soothing device according to the operation intention of the user; and
a soothing structure, comprising a control board and a vibration module, wherein the control board is respectively connected to the stepless adjustment structure and the vibration module, and is configured to control the vibration module to vibrate, stop vibration, and change of vibration frequency.

2. The soothing device according to claim 1, wherein the stepless adjustment structure comprises a switch and a stepless converter, the switch is connected to the stepless converter, the switch is configured with an operation portion, and the stepless converter is configured to receive a operation instruction applied to the operation portion by a user and convert the operation instruction into a corresponding stepless adjustment signal.

3. The soothing device according to claim 2, wherein the switch comprises an adjustment knob capable of being adjusted along a first direction or a second direction, and the adjustment knob rotated in the first direction is configured to turn on the vibration module and/or adjust the vibration frequency of the vibration module, and the adjustment knob rotated in the second direction is configured to turn off the vibration module and/or adjust the vibration frequency of the vibration module, wherein the first direction is opposite to the second direction.

4. The soothing device according to claim 1, wherein the stepless adjustment structure comprises a remote device and a wireless connection module, the remote device communicates with the wireless connection module, and after the wireless connection module receives an operation instruction from a user, the control board instructs the vibration module to vibrate at a corresponding frequency based on the operation instruction.

5. The soothing device according to claim 1, wherein the stepless adjustment structure comprises a sensor connected to the control board to collect a heartbeat information and provide a corresponding vibration frequency signal to the control board.

6. The soothing device according to claim 1, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

7. The soothing device according to claim 6, further comprising:
a timing structure, connected to the control board, and at least a part of the timing structure exposed to the mounting shell.

8. The soothing device according to claim 1, further comprising:
a battery structure connecting the stepless adjustment structure and the soothing structure, and configured to provide electrical energy.

9. The soothing device according to claim 8, further comprising:
a charging interface connecting the control board and the battery structure.

10. The soothing device according to claim 1, further comprising:
an indicator light connected to the control board.

11. The soothing device according to claim 2, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

12. The soothing device according to claim 11, further comprising:
a timing structure, connected to the control board, and at least a part of the timing structure exposed to the mounting shell.

13. The soothing device according to claim 3, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

14. The soothing device according to claim 13, further comprising:
a timing structure, connected to the control board, and at least a part of the timing structure exposed to the mounting shell.

15. The soothing device according to claim 4, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

16. The soothing device according to claim 15, further comprising:
a timing structure, connected to the control board, and at least a part of the timing structure exposed to the mounting shell.

17. The soothing device according to claim 5, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

18. The soothing device according to claim 17, further comprising:
a timing structure, connected to the control board, and at least a part of the timing structure exposed to the mounting shell.

19. The soothing device according to claim 8, further comprising:
a mounting shell comprising an upper shell and a lower shell, wherein the upper shell and the lower shell are combined to form a receiving cavity inside, and the receiving cavity is configured to receive the soothing structure and the stepless adjustment structure.

20. The soothing device according to claim 19, further comprising:
a timing structure, connected to the control board, and at least a part of the timing structure exposed to the mounting shell.
